(19) United States
(12) Patent Application Publication
(10) Pub. No.: US 2011/0003837 A1
(43) Pub. Date: Jan. 6, 2011

(54) MODIFIED RELEASE FORMULATIONS OF HMG COA REDUCTASE INHIBITORS

(75) Inventors: Sheetal KULKARNI, Pune (IN); Srirupa DAS, Pune (IN); Ashish Ashokrao DESHMUKH, Pune (IN); Satish Kumar DALAL, Pune (IN); Shirishkumar KULKARNI, Pune (IN)

Correspondence Address: PAULY, DEVRIES SMITH & DEFFNER, L.L.C., Plaza VII-Suite 3000, 45 South Seventh Street, MINNEAPOLIS, MN, 55402-1630, US

(73) Assignee: LUPIN LIMITED, Mumbai 400 098 Maharashtra (IN)

(21) Appl. No.: 12/865,448

(22) PCT Filed: Jan. 30, 2009
(86) PCT No.: PCT/IN09/00069

§ 371 Date: Jul. 30, 2010

(30) Foreign Application Priority Data
Jan. 30, 2008 (IN) 145/KOL/2008

(51) Int. Cl. A61K 31/505; A61P 3/06

(52) U.S. Cl. 514/275

(57) ABSTRACT

Modified release formulations of HMG Co-A reductase inhibitors, which provide reduced incidence of rhabdomyolysis, renal toxicity and other side effects by increasing hepatic bioavailability and decreasing systemic availability upon oral administration. The modified release pharmaceutical formulation comprises a therapeutically effective amount of HMG CoA reductase inhibitor or a pharmaceutically acceptable salt(s), polymorph(s), solvate(s), hydrate(s), prodrug or metabolite thereof, one or more release modifying agent(s) and one or more pharmaceutically acceptable excipient(s), wherein the modified release formulation provides reduced incidence of adverse effects and improved efficacy when compared to the immediate release formulation upon oral administration.

### FIELD OF THE INVENTION

The present invention is directed towards modified release formulations of HMG Co-A reductase inhibitors, which provide reduced incidence of rhabdomyolysis, renal toxicity and other side effects by increasing hepatic bioavailability and decreasing systemic availability upon oral administration.

More particularly the present invention relates to modified release formulations of rosuvastatin or a pharmaceutically acceptable salt(s), polymorph(s), solvate(s), hydrate(s), prodrug or metabolite thereof, which provides reduced incidence of rhabdomyolysis and renal toxicity upon oral administration.

### BACKGROUND OF THE INVENTION

Atherosclerosis and its various clinical presentations as coronary artery disease, cerebrovascular disease, peripheral vascular disease and other conditions, is a major cause of death in western countries. Hypercholesterolemia is a primary risk factor for death from these conditions.

HMG CoA reductase inhibitors or statins are a class of compound that competitively inhibit 3-hydroxy-3-methylglutaryl-coenzyme A (HMG-CoA) reductase, which catalyse the conversion of HMG CoA to mevalonate, an early rate limiting step in cholesterol biosynthesis. Inhibitors of HMG-CoA reductase have proved to be most effective in reducing the plasma levels of cholesterol in patients with both hypercholesterolemia and normocholesterolemia. These drugs lower cholesterol by slowing down the production of cholesterol and by increasing the liver's ability to remove the LDL-cholesterol already in the blood. The large reductions in total and LDL-cholesterol produced by these drugs resulted in large reductions in heart attacks and heart disease deaths. HMG CoA reductase inhibitors also produce a modest increase in HDL-cholesterol and reduce elevated triglyceride levels. For example, simvastatin in clinical trials reduced cholesterol and LDL cholesterol by 25% and 35% respectively. Simvastatin was reported in trials to reduce the risk of a major coronary event by 34%. Statins have become the drugs most often prescribed when a person with heart disease needs a cholesterol-lowering medicine.

Rosuvastatin 40 mg daily has been shown in clinical trials to provide the greatest LDL-C reduction (up to 63%) compared to other statins.

The HMG CoA reductase inhibitors available in market are Atorvastatin (Lipitor®), Fluvastatin (Lescol® and Lescol XL®), Lovastatin (Altoprev®) Pravastatin (Pravachol®)), Rosuvastatin (Crestor®), Simvastatin (Zocor®),) and Pitvastatin.

However, the treatment of patients with inhibitors of HMG-CoA reductase, such as the statins, is accompanied by adverse side effects, which cause discomfort and may necessitate discontinuation of medication. As HMG-CoA reductase inhibitors are often used as a long term means for prevention of heart disease in patients who may be otherwise healthy, there is a need for a method of treatment of hypercholesterolemia without the associated adverse effects of HMG-CoA reductase inhibitors. Adverse effects known to be associated with the use of HMG-CoA reductase inhibitors include muscle cramps, myalgia, increased risk of myopathy, transient elevation of creatine phosphokinase levels from skeletal muscle, and even rhabdomyolysis. Rhabdomyolysis is the breakdown of muscle fibers resulting in the release of muscle fiber contents into the circulation. Some of these are toxic to the kidney and frequently result in kidney damage. Crestor®, Liptior, Zocor, Mevacor, Pravachol, and Lescol are believed to increase the risk of rhabdomyolysis. The risk is significantly increased by concomitant use of drugs that inhibit metabolism of HMG CoA reductase inhibitor. As a result systemic levels of HMG CoA Reductase Inhibitor are increased resulting in greater toxicity. The risk of these side effects is further increased when some other lipid lowering drugs, for example, gemfibrizol, are co-prescribed. Occasionally, HMG CoA reductase inhibitors use causes an increase in liver enzymes. Because liver problems may develop without symptoms, people who take HMG CoA reductase inhibitors should have their liver function tested periodically.

HMG CoA reductase inhibitors may cause muscle pain and tenderness (statin myopathy). In severe cases, muscle cells can break down (rhabdomyolysis) and release a protein called myoglobin into the bloodstream. Myoglobin can impair kidney function and lead to kidney failure. Certain drugs when taken with HMG CoA reductase inhibitors can increase the risk of rhabdomyolysis. These include gemfibrozil, erythromycin (Erythrocin), anti fungal medications, nefazodone (Serzone), cyclosporine and niacin. The most common side effects include: nausea, diarrhea, constipation, muscle aching. In addition to the side effects listed above there are serious side effects such as rhabdomyolysis (which may be fatal) and renal impairment associated with HMG CoA reductase inhibitors medications.

Further, the use of HMG-CoA reductase inhibitors has also been reported to aggravate cardiac function and uncommonly, to worsen cardiac failure. These adverse effects in both skeletal muscle and the heart, are not common, but appear to have a common pathway related to inhibition of the synthesis of ubiquinone. Depletion of Coenzyme Q10 in skeletal and cardiac muscle has been linked to the development of both skeletal myopathy and cardiac myopathy, to the development of fatigue, and has been proposed as the mechanism of action of statin-induced muscle disease. Since fatigue is a widely reported symptom in patients with cardiovascular disease, many of whom are taking HMG-CoA reductase inhibitors for the treatment of hypercholesterolemia, it is likely that a contribution to the cause of fatigue by these drugs has not been appreciated and is therefore under-diagnosed.

Rosuvastatin is metabolized slowly in the liver, where metabolism by cytochrome P450 isoenzymes is limited. Although one major N-desmethyl metabolite (formed primarily by CYP2C9 and CYP2C19) has been identified, it is seven-fold less active than the parent compound in inhibiting HMG-CoA reductase. Furthermore, it is believed that 90% of the inhibitory activity of rosuvastatin is due to the parent compound. Rosuvastatin is selectively taken up into hepatocytes based on a carrier-mediated mechanism, with up to 90% of the absorbed dose extracted by the liver. Although the presence of food decreases the rate of absorption, the overall extent of absorption remains constant. Peak plasma concentrations (Cₘₐₓ), as well as the AUC, show a relatively linear relationship with respect to doses ranging from 5 to 80 mg, with a Tmax that ranges from 3 to 5 hours. Furthermore, rosuvastatin has a long elimination half-life (t½) of 20 hours. Clearance of rosuvastatin occurs mainly through biliary excretion (90%), while 10% is excreted in the urine.

Unlike pravastatin (but like atorvastatin), rosuvastatin is stable in acidic environments like that found in the stomach. Once rosuvastatin passes out of the stomach, it is believed to enter the circulation via a carrier-mediated transport mechanism in the small intestine. Following absorption, rosuvastatin enters hepatocytes through a carrier-mediated transport mechanism. The organic anion transport polypeptide-C, which is expressed at high levels in hepatocytes, is thought to play a key role in selectively delivering rosuvastatin to the HMG-CoA reductase target enzyme in the liver. Accordingly, the amount of rosuvastatin that is ultimately absorbed by the liver and available for binding to HMG-CoA reductase depends on the rates of uptake in the intestine and liver. In case of rosuvastatin the development of severe myopathy or rhabdomyolysis requiring hospitalization for IV hydration occurred at an incidence only at the 80 mg dose.

Researchers estimate that between 1% and 5% of statin users will experience muscle pain and weakness as a side effect.

The incidence of myopathy, in clinical trials, of rosuvastatin 5 to 40 mg was between 0.1 to 0.2%, which are similar to, rates seen with other currently approved statins. The risk of muscle and renal toxicity appear dose related and are clearly evident at 80 mg dose. Rosuvastatin levels more than 50 ng/ml in plasma developed muscle and or renal toxicity. Only a few patients treated with 40 mg rosuvastatin (2%) had drug levels within this range and greater proportion of patients treated with 80 mg (33%) achieved drug levels greater than 50 ng/ml. Further in certain clinical situations which may increase drug level require careful consideration as patients in these settings may be exposed to drug levels beyond what is typical for the 20 and 40 mg doses.

Several studies were carried out to study the effect of statins on myopathic events. One such study shows that out of 32,225 patients identified in study to estimate the prevalence of myopathic events, particularly myalgia, myositis, and rhabdomyolysis were diabetics (n=10,247) and nondiabetics (n=21, 978). A greater proportion of statin initiators in both the diabetes (7.9% vs 5.5%; P<0.001) and nondiabetes cohorts (9.0% vs 3.7%; P<0.001) experienced myopathic events. The prevalence of severe myositis was 0.4 per 1000 person-years (95% CI, 0.2-0.7) and 0.8 per 1000 person-years (95% CI, 0.6-1.1) among statin initiators with or without diabetes, respectively. Another study quantified the risk of myositis associated with statin and fibrate drug. Myositis was significantly associated with statin monotherapy (RR 2.8 [95% confidence interval, CI=1.3-5.9]), statin-fibrate combination therapy (9.1 [95% CI=3.5-23]), comorbid liver disease (4.3 [95% CI=1.5-13], and/or renal disease (2.5 [95% CI=1.3-5.0]).

Yet another study was undertaken to identify and characterize risk factors for rhabdomyolysis in patients' prescribed statin monotherapy or statin plus fibrate therapy. Statin users 65 years of age and older have four times the risk of hospitalization for rhabdomyolysis than those under age 65 (odds ratio (OR)=4.36, 95% confidence interval (CI): 1.5, 14.1).

In one another studies, of 866 total reported cases, 482 (56%) were associated with monotherapy and 384 (44%) related to combination therapy with fabric acid. More than 80% of reported cases for each drug resulted in hospitalization for renal failure and dialysis. 80 patients expired from events related directly to rhabdomyolysis. Reporting rates for all statins, except for cerivastatin, were similar and much lower than 1 per 100,000 prescriptions.

Delayed release statin formulation are described in US 2005/0119331, applicant Butler et al, relates to method of increasing bioavailability of acid stable, carrier mediated statins. The formulation results in, delayed release of substantial amount of statin until the composition has passed out of the stomach and than releases the statin at a rate that avoids saturating the intestinal and hepatocyctic mechanisms.

US 2002/0160044, applicant Howard J Smith and associates, relates to methods of drug treatment where liver or portal venous circulation is the primary therapeutic target and in particular to methods of treatment or prevention of diseases that are selective for the liver and there by minimize side effects. The method involves use of a slow release formulation of a low dose of HMG CO A reductase inhibitor that is itself metabolized by the liver.

US 2005/0239884, applicant Novartis, relates to pharmaceutical compositions for sustained release comprising active ingredient an HMG CoA reductase inhibitor or pharmaceutically acceptable salt, said composition comprising an inner phase and an outer phase wherein at least the outer phase comprises at least one matrix former.

US 2005/0203186, applicant Ratio harm, relates to medicament containing at least one active ingredient, which lowers the cholesterol in the blood, characterized in that it has means for providing release characteristics for the active ingredient with which the active ingredient is released with at least two different release rates.

One way of avoiding/reducing the side effects associated with immediate release statins is the use of modified release formulation. The present invention provides administering statins in a manner that is selective for the liver, and that will reduce hypercholesterolemia without systemic depression of Coenzyme Q10. Following ingestion, statins are absorbed through the intestine into the hepatic portal vein and distributed into the liver, which is the primary site of action and the primary site of cholesterol synthesis. The formulation slowly releases the HMG CoA reductase inhibitor such that clinically effective levels of the HMG-CoA reductase inhibitor will be achieved in the liver, but the systemic blood levels will be low and thus will have reduced adverse effects and improved efficacy.

Thus, there exists a need in the art, for new formulations that allow for more optimal absorption of the statins in the intestine and in the liver. Such modified-release formulations would help maximize statin absorption in the intestine and liver, and thus limit systemic exposure and the associated adverse effects.

While a few patent applications described above disclose the extended release formulations of statin, but none of these explain the need for maintaining low systemic plasma concentration of statin as compared to the immediate release formulation. This is achieved by reducing systemic exposure of rosuvastatin in the systemic circulation. It is also contemplated that the present formulation reduces Cₘₐₓ and AUC compared to immediate release formulation. This would further lead to reduction in the adverse effect and will lead to improved efficacy.

Further, the formulation of present invention increases hepatic bioavailability and decreases systemic exposure of HMG CoA reductase inhibitor.

### OBJECTS OF THE INVENTION

One object of the present invention is a modified release pharmaceutical formulation comprising a therapeutically effective amount of HMG CoA reductase inhibitor or a pharmaceutically acceptable salt(s), polymorph(s), solvate(s), hydrate(s), prodrug or metabolite thereof, one or more release modifying agent(s) and one or more pharmaceutically acceptable excipients (s), wherein the modified release formulation provides reduced incidence of adverse effects and improved efficacy when compared to the immediate release formulation upon oral administration.

Another object of the present invention is a modified release pharmaceutical formulation comprising a therapeutically effective amount of rosuvastatin or a pharmaceutically acceptable salt(s), polymorph(s), solvate(s), hydrate(s), prodrug or metabolite thereof, one ore more release modifying agent(s) and one or more pharmaceutically acceptable excipient(s), wherein Cₘₐₓ of modified release formulation is statistically lower than the immediate release formulation upon oral administration.

Another object of the present invention is a modified release pharmaceutical formulation comprising a therapeutically effective amount of rosuvastatin or a pharmaceutically acceptable salt(s), polymorph(s), solvate(s), hydrate(s), prodrug or metabolite thereof, one or more release modifying agent(s) and one or more pharmaceutically acceptable excipient(s), wherein Tₘₐₓ of, modified release formulation is statistically longer than the immediate release formulation upon oral administration.

Another object of the present invention is a modified release pharmaceutical formulation comprising a therapeutically effective amount of rosuvastatin or a pharmaceutically acceptable salt(s), polymorph(s), solvate(s), hydrate(s), prodrug or metabolite thereof, one or more release modifying agent(s) and one or more pharmaceutically acceptable excipient(s), wherein AUC of modified release formulation is lower than the immediate release formulation upon oral administration.

Another object of the present invention is a modified release pharmaceutical formulation comprising a therapeutically effective amount of rosuvastatin or a pharmaceutically acceptable salt(s), polymorph(s), solvate(s), hydrate(s), prodrug or metabolite thereof, one or more release modifying agent(s) and one or more pharmaceutically acceptable excipient(s), wherein the modified release formulation produces increased hepatic bioavailability and decreased systemic bioavailability when compared to the immediate release formulation upon oral administration.

Another object of the present invention is a modified release pharmaceutical formulation comprising a therapeutically effective amount of rosuvastatin or a pharmaceutically acceptable salt(s), polymorph(s), solvate(s), hydrate(s), prodrug or metabolite thereof, one ore more release modifying agent(s) and one or more pharmaceutically acceptable excipient(s), wherein the systemic exposure of rosuvastatin is reduced when compared to the immediate release formulation upon oral administration.

Another object of the present invention is a modified release pharmaceutical formulation comprising a therapeutically effective amount of rosuvastatin or a pharmaceutically acceptable salt(s), polymorph(s), solvate(s), hydrate(s), prodrug or metabolite thereof, one or more release modifying agent(s) and one or more pharmaceutically acceptable excipient(s), wherein the composition upon oral administration to a mammal in need thereof releases rosuvastatin in a controlled manner thereby minimizing or avoiding the first peak as compared to the two peaks observed with immediate release formulation.

Another object of the present invention is a modified release pharmaceutical formulation comprising a therapeutically effective amount of rosuvastatin or a pharmaceutically acceptable salt(s), polymorph(s), solvate(s), hydrate(s), prodrug or metabolite thereof, one or more release modifying polymer(s) and one or more pharmaceutically acceptable excipient(s); wherein about 10-50% of drug is released in about 2 hours, about 20-70% of drug is released in about 4 hours, about 40-90% of drug is released in about 8 hrs, and more than about 75% of drug is released in about 24 hours.

### DETAILED DESCRIPTION OF INVENTION

For the present invention HMG COA reductase inhibitor and statin are used interchangeably.

HMG-CoA reductase inhibitors are the most commonly prescribed drugs for lowering cholesterol levels for long-term use. The adverse effects of HMG CoA reductase inhibitors are headaches, nausea and fever and the major effects are muscle pain and muscle disease and serious liver problems.

Statins lower blood lipid levels by reducing cholesterol biosynthesis in the liver. Accordingly, statins are known for their ability to help reduce levels of total cholesterol and low-density-lipoprotein cholesterol, which is of primary importance in preventing coronary heart disease. Because of possible unwanted effects in non-liver tissues, systemic availability of statins is considered undesirable. Furthermore, to increase the level of HMG-CoA reductase inhibition, it is desirable to maximize hepatic bioavailability.

Rosuvastatin is another new member of the statin family whose uptake is governed by carrier-mediated transport mechanisms. Rosuvastatin (which recently received FDA approval under the name CRESTOR® is a fully synthetic single enantiomeric hydroxy acid, which belongs to a novel series of N-methanesulfonamide pyrimidine and N-methanesulfonyl pyrrole-substituted 3,5-dihydroxy-6-heptenoates (Cheng-Lai (2003) Heart Disease 5:72). Although rosuvastatin shares the common statin pharmacophore, it has an additional methane-sulfonamide group that increases its hydrophilicity. Because of its increased hydrophilic character and its large molecular size (MW 1001 as the bis calcium salt; MW 480 as the free acid) rosuvastatin has difficulty crossing biological membranes. Rosuvastatin is also relatively poorly soluble in water under both acidic and basic conditions. For example, as defined by the U.S. Pharmacopeia (2002), rosuvastatin is considered “sparingly soluble.” Further it has been found that HMG CoA reductase inhibitors undergo enterohepatic recirculation due to which the plasma concentration vs time profile shows multiple peaks depicting that first peak occurs due to the rapid release of drug in plasma further the another peak occurs after enterohepatic recirculation.

We have surprisingly found that the modified release formulations of the present invention reduce the total systemic exposure of rosuvastatin in systemic circulation when compared to the immediate release formulation upon oral administration.

Thus, the modified release formulations of the present invention eliminates the multiple peaks as in immediate releases formulation and thereby increases hepatic bioavailability and then reducing the adverse effects when compared to immediate release formulations.

The present invention also relates to modified release HMG CoA reductase inhibitors formulation producing blood plasma levels over 24 hours after ingestion comprising therapeutically effective amount of statin or a pharmaceutically acceptable salt(s), polymorph(s), solvate(s), hydrate(s), prodrug or metabolite thereof, one or more release modifying agent(s) and one or more pharmaceutically acceptable excipients.

Modified release formulation describes a formulation that does not release active drug substance immediately after oral dosing and that allows a reduction in dosage frequency. A modified release formulation includes but is not limited to extended release, delayed release, sustained release formulations or controlled release or timed release. Further gastroretentive or bioadhesive formulation will also be included within the scope of present invention. A controlled release formulation may be administered once a day. Release modifying polymers according to present invention includes agents, which controls the release of drug from the formulation.

“Therapeutically effective amount” means that the amount of active agent, which halts or reduces the progress of the condition being treated or which otherwise completely or partly cures or acts palliatively on the condition. A person skilled in the art can easily determine such an amount by routine experimentation and with an undue burden.

“Optional” or “optionally” means that the subsequently described circumstance may or may not occur, so that the description includes instances where the circumstance occurs and instances where it does not.

By “pharmaceutically acceptable” is meant a carrier comprised of a material that is not biologically or otherwise undesirable.

“Cₘₐₓ” as used herein, means maximum plasma concentration of the statin produced by the ingestion of the composition of the invention or the marketed Crestor® formulation. Cₘₐₓ or peak concentration may be used interchangeably.

A drug can have more than one peak; one may be due to systemic absorption and other due to enterohepatic recirculation. It has been known that rosuvastatin shows two peaks one due to systemic absorption and another due to enterohepatic recirculation.

“Tₘₐₓ” as used herein, means time to the maximum observed plasma concentration.

“AUC” as used herein, means area under the plasma concentration-time curve.

“Adverse effects” as used herein, means those physiological effects to various systems in the body, which cause discomfort to the individual subject.

“Bioadhesion” is defined as the ability of a material to adhere to a biological tissue for an extended period of time. Bioadhesion is one solution to the problem of inadequate residence time resulting from stomach emptying and intestinal peristalsis, and from displacement by ciliary movement. For sufficient bioadhesion to occur, an intimate contact must exist between the bioadhesive and the receptor tissue, the bioadhesive must penetrate into the crevice of the tissue surface and/or mucus, and mechanical, electrostatic, or chemical bonds must form. Bioadhesive properties of polymers are affected by both the nature of the polymer and by the nature of the surrounding media.

Bioadhesive and mucoadhesive can be used interchangeably.

For purposes of this invention, residence time is the time required for a pharmaceutical dosage form to transit through the stomach to the rectum i.e. the pharmaceutical dosage forms of the invention may have an increased retention time in the stomach and/or small and/or large intestine, or in the area of the gastrointestinal tract that absorbs the drug contained in the pharmaceutical dosage form. For example, pharmaceutical dosage forms of the invention can be retained in the small intestine (or one or two portions thereof, selected from the duodenum, the jejunum and the ileum). These pharmaceutical dosage forms as a whole may include a bioadhesive polymeric coating that is applied to at least one surface of the dosage form.

The HMG CoA reductase inhibitors or statin, which may be used in the present invention, includes but not limited to Lovastatin, pravastatin, atorvastatin, nivasatin, rosuvastatin, cerivastatin, pitvastatin and others. The most preferred statin is rosuvastatin.

Release modifying polymers that are used in the present formulation may include hydrophilic polymer, hydrophobic polymer or a combination of hydrophilic and hydrophilic polymers.

Examples of suitable hydrophilic polymers include but not limited to hydroxypropyl methylcellulose, hydroxypropyl cellulose, methylcellulose, vinyl acetate copolymers, polysaccharides as alginates, xanthan gum, Chitosan, carrageenan, dextran and the like, polyalkylene oxides as polyethylene oxide and the likes, methaacrylic acid copolymers, maleic anhydride/methyl vinyl ether copolymers and the like. Hydrophobic polymers include acrylates, cellulose derivatives as ethylcellulose, cellulose acetate and the likes, methaacrylates, high molecular weight polyvinyl alcohols, waxes and the likes.

The polymers used can also be eroding or non-eroding or combination of both.

The polymers, which may be used for bioadhesion, are described below.

Natural polymers include but are not limited to proteins (e.g., hydrophilic proteins), such as pectin, zein, modified zein, casein, gelatin, gluten, serum albumin, or collagen, chitosan, oligosaccharides and polysaccharides such as cellulose, dextrans, tamarind seed polysaccharide, gellan, carrageenan, xanthan gum, gum Arabic; hyaluronic acid, polyhyaluronic acid, alginic acid, sodium alginate.

When the bioadhesive polymer is a synthetic polymer, the synthetic polymer is typically selected from but are not limited to polyamides, polycarbonates, polyalkylenes, polyalkylene glycols, polyalkylene oxides, polyalkylene terephthalates, polyvinyl alcohols, polyvinyl ethers, polyvinyl esters, polyvinyl halides, polyvinylpyrrolidone, polyglycolides, polysiloxanes, polyurethanes, polystyrene, polymers of acrylic and methacrylic esters, polylactides, poly(butyric acid), poly(valeric acid), poly(lactide-co-glycolide), polyanhydrides, polyorthoesters, poly(fumaric acid), poly(maleic acid), and blends and copolymers or mixtures thereof.

Other polymers suitable for use in the invention include, but are not limited to, methyl cellulose, ethyl cellulose, hydroxypropyl cellulose, hydroxypropylmethyl cellulose, hydroxybutylmethyl cellulose, cellulose acetate, cellulose propionate, cellulose acetate butyrate, cellulose acetate phthalate, carboxymethyl cellulose, cellulose triacetate, cellulose sulfate sodium salt, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate) polyethylene, polypropylene, poly(ethylene glycol), poly(ethylene oxide), poly(ethylene terephthalate), polyvinyl acetate), polyvinyl chloride, polystyrene, polyvinyl pyrrolidone, and polyvinylphenol. Polylactides, polyglycolides and copolymers thereof, poly(ethylene terephthalate), poly(butyric acid), poly(valeric acid), poly(lactide-co-caprolactone), poly[lactide-co-glycolide], polyanhydrides (e.g., poly(adipic anhydride)), polyorthoesters, blends and copolymers thereof.

Another group of polymers suitable for use as bioadhesive polymers but not necessarily limited to polymers having a hydrophobic backbone with at least one hydrophobic group pendant from the backbone. Suitable hydrophobic groups are groups that are generally non-polar. Examples of such hydrophobic groups include alkyl, alkenyl and alkynyl groups. Preferably, the hydrophobic groups are selected to not interfere and instead to enhance the bioadhesiveness of the polymers.

A further group of polymers suitable for use as bioadhesive polymers but not necessarily limited to polymers having a hydrophobic backbone with at least one hydrophilic group pendant from the backbone. Suitable hydrophilic groups include groups that are capable of hydrogen bonding or electrostatically bonding to another functional group. Example of such hydrophilic groups include negatively charged groups such as carboxylic acids, sulfonic acids and phosphonic acids, positively charged groups such as (protonated) amines and neutral, polar groups such as amides and imines.

Preferably, the hydrophilic groups are selected to not to interfere and instead to enhance the bioadhesiveness of the polymers. In embodiments of the present invention, a pharmaceutical composition comprises an active agent and at least one swellable polymer.

Swellable polymers include, but are not limited to, a crosslinked poly(acrylic acid), a poly(alkylene oxide), a polyvinyl alcohol), a polyvinyl pyrrolidone); a polyurethane hydrogel, a maleic anhydride polymer, such as a maleic anhydride copolymer, a cellulose polymer or derivatives thereof, a polysaccharide, starch, and starch based polymers.

Polymers can be modified by increasing the number of carboxylic groups accessible during biodegradation, or on the polymer surface. The polymers can also be modified by binding amino groups to the polymer. The polymers can be modified using any of a number of different coupling chemistries available in the art to covalently attach ligand molecules with bioadhesive properties to the surface-exposed molecules of the polymeric microspheres.

Lectins can be covalently attached to polymers to render them target specific to the mucin and mucosal cell layer. The attachment of any positively charged ligand, such as polyethyleneimine or polylysine, to a polymer may improve bioadhesion due to the electrostatic attraction of the cationic groups coating the beads to the net negative charge of the mucus. The mucopolysaccharides and mucoproteins of the mucin layer, especially the sialic acid residues, are responsible for the negative charge coating. Any ligand with a high binding affinity for mucin could also be covalently linked to most polymers with the appropriate chemistry, such as with carbodiimidazole (CDI), and be expected to influence the binding to the gut. For example, polyclonal antibodies raised against components of mucin or else intact mucin, when covalently coupled to a polymer, would provide for increased bioadhesion. Similarly, antibodies directed against specific cell surface receptors exposed on the lumenal surface of the intestinal tract would increase the residence time when coupled to polymers using the appropriate chemistry. The ligand affinity need not be based only on electrostatic charge, but other useful physical parameters such as solubility in mucin or specific affinity to carbohydrate groups.

The covalent attachment of any of the natural components of mucin in either pure or partially purified form to the polymers generally increases the solubility of the polymer in the mucin layer. The list of useful ligands include but are not limited to the following: sialic acid, neuraminic acid, n-acetyl-neuraminic acid, n-glycolylneuraminic acid, 4-acetyl-n-acetylneuraminic acid, diacetyl-n-acetylneuraminic acid, glucuronic acid, iduronic acid, galactose, glucose, mannose, fucose, any of the partially purified fractions prepared by chemical treatment of naturally occurring mucin, e.g., mucoproteins, mucopolysaccharides and mucopolysaccharide-protein complexes, and antibodies immunoreactive against proteins or sugar structure on the mucosal surface.

The attachment of polyamino acids containing extra pendant carboxylic acid side groups, such as polyaspartic acid and polyglutamic acid, may also increase bioadhesiveness. The polyamino chains would increase bioadhesion by means of chain entanglement in mucin strands as well as by increased carboxylic charge.

The formulation will, in general comprise of one or more excipients. Examples of pharmaceutically acceptable excipients include, but are not limited to, diluents, disintegrants, lubricant, glidant, binders, fillers, surfactant, solubilizers, wetting agents, chelating agents, stabilizers, alkalizing agents or amino acids. A combination of excipients may also be used. The amount of excipient(s) employed will depend upon how much active agent is to be used. One excipient can perform more than one function.

Binders include, but are not limited to, starches such as potato starch, wheat starch, corn starch; microcrystalline cellulose such as products known under the registered trade marks Avicel, Filtralc, Heweten or Pharmacel; celluloses such as hydroxypropyl cellulose, hydroxyethyl cellulose, hydroxypropylmethyl cellulose (HPMC), ethyl cellulose, sodium carboxy methyl cellulose; natural gums like acacia, alginic acid, guar gum; liquid glucose, dextrin, povidone, syrup, polyethylene oxide, polyvinyl pyrrolidone, poly-N-vinyl amide, polyethylene glycol, gelatin, poly propylene glycol, tragacanth, combinations there of and other materials known to one of ordinary skill in the art and mixtures thereof.

Fillers or diluents, which include, but are not limited to confectioner's sugar, compressible sugar, dextrates, dextrin, dextrose, fructose, lactitol, mannitol, sucrose, starch, lactose, xylitol, sorbitol, talc, microcrystalline cellulose, calcium carbonate, calcium phosphate dibasic or tribasic, calcium sulphate, and the like can be used.

Lubricants may be selected from, but are not limited to, those conventionally known in the art such as Mg, Al or Ca or Zn stearate, polyethylene glycol, glyceryl behenate, mineral oil, sodium stearyl fumarate, stearic acid, hydrogenated vegetable oil and talc.

Glidants include, but are not limited to, silicon dioxide; magnesium trisilicate, powdered cellulose, starch, talc and tribasic calcium phosphate, calcium silicate, magnesium silicate, colloidal silicon dioxide, silicon hydrogel and other materials known to one of ordinary skill in the art.

The present formulations may optionally contain a surface-active agent. Unlimiting examples of surfactants include water-soluble or water dispersible nonionic, semi-polar nonionic, anionic, cationic, amphoteric, or zwitterionic surface-active agents; or any combination thereof, these include but not limited to poloaxmer, dioctyl sodium sulfosuccinate (DSS), triethanolamine, sodium lauryl sulphate (SLS), polyoxyethylene sorbitan and poloxalkol derivatives, quaternary ammonium salts or other pharmaceutically acceptable surface-active agents known to one ordinary skilled in the art.

The pharmaceutical formulation according to the present invention include but is not limited to tablets (single layered tablets, multilayered tablets, mini tablets, bioadhesive tablets, caplets, matrix tablets, tablet within a tablet, mucoadhesive tablets, modified release tablets, pulsatile release tablets, timed release tablets), pellets, beads, granules, sustained release formulations, capsules, microcapsules, tablets in capsules and microspheres, matrix formulations, microencapsulation and powder/pellets/granules for suspension.

The pharmaceutical dosage form of the invention can optionally have one or more coatings such as film coating, sugar coating, enteric coating, bioadhesive coating and other coatings known in the art. These coatings help pharmaceutical formulations to release the drug at the required site of action. In one example, the additional coating prevents the dosage from contacting the mouth or esophagus. In another example, the additional coating remains intact until reaching the small intestine (e.g., an enteric coating). Premature exposure of a bioadhesive layer or dissolution of a pharmaceutical dosage form in the mouth can be prevented with a layer or coating of hydrophilic polymers such as HPMC or gelatin. Optionally, Eudragit FS 30D or other suitable polymer may be incorporated in coating composition to retard the release of the drug to ensure drug release in the colon.

These coating layers comprises one or more excipients selected from the group comprising coating agents, opacifiers, taste-masking agents, fillers, polishing agents, colouring agents, antitacking agents and the like.

Coating agents which are useful in the coating process, include, but are not limited to, polysaccharides such as maltodextrin, alkyl celluloses such as methyl or ethyl cellulose, hydroxyalkylcelluloses (e.g. hydroxypropylcellulose or hydroxypropylmethylcelluloses); polyvinylpyrrolidone, acacia, corn, sucrose, gelatin, shellac, cellulose acetate pthalate, lipids, synthetic resins, acrylic polymers, opadry, polyvinyl alcohol (PVA), copolymers of vinylpyrrolidone and vinyl acetate (e.g. marketed under the brand name of Plasdone) and polymers based on methacrylic acid such as those marketed under the brand name of Eudragit. These may be applied from aqueous or non-aqueous systems or combinations of aqueous and non-aqueous systems as appropriate. Additives can be included along with the film formers to obtain satisfactory films. These additives can include plasticizers such as dibutyl phthalate, triethyl citrate, polyethylene glycol(PEG) and the like, antitacking agents such as talc, stearic acid, magnesium stearate and colloidal silicon dioxide and the like, surfactants such as polysorbates and sodium lauryl sulphate, fillers such as talc, precipitated calcium carbonate, Polishing agents such as Beeswax, carnauba wax, synthetic chlorinated wax and opacifying agents such as titanium dioxide and the like. All these excipients can be used at levels well known to the persons skilled in the art.

Pharmaceutical dosage forms of the invention can be coated by a wide variety of methods. Suitable methods include compression coating, coating in a fluidized bed or a pan and hot melt (extrusion) coating. Such methods are well known to those skilled in the art.

Non-permeable coatings of insoluble polymers, e.g., cellulose acetate, ethylcellulose, can be used as enteric coatings for delayed/modified release (DR/MR) by inclusion of soluble pore formers in the coating, e.g., PEG, PVA, sugars, salts, detergents, triethyl citrate, triacetin, etc.

Multi-layer or gradient tablets can be assembled in several different ways.

In one embodiment, the tablet comprises at least one solid core and two outer layers, each comprising one or more pharmaceutical polymers and/or pharmaceutical excipients. The core comprises active ingredient and release modifying polymer (s). The two outer layers are bioadhesive.

In another embodiment, the tablet comprises at least one core and two outer layers, each comprising drug and one or more pharmaceutical polymers and/or pharmaceutical excipients. Such tablets can also be used to commence release of different drugs at different times, by inclusion of different drugs in separate layers.

In another embodiment, the multi-layer tablet consists of a core and two outer layers, each comprising a drug and one or more pharmaceutical polymers or pharmaceutical excipients, wherein at least one polymer or excipient is hydrophobic.

In another preferred embodiment the present invention relates to formulation which consists of multilayer tablet wherein at least one layer consist of a controlled release polymer and the active ingredient and at least one layer which consist of bioadhesive polymer, where each layer includes one or more excipients.

In another embodiment the present invention relates to formulation which consists of multilayer tablet wherein at least one layer consist of a controlled release polymer and at least one layer which consist of bioadhesive polymer, where each layer includes one or more excipients and drug.

Further, the modified release formulation of statin may include an immediate release layer. Also combination of statin with other lipid lowering agents such as other statins, fabric acid derivatives, bile acid sequestrants, niacin, HDL increasing agents such as omega 3 fatty acids, or anti-hypertensive agents or PDE-5 inhibitors such as Sildenafil, platelet inhibiting agents as aspirin and other agents also fall within the scope of the invention. These other agents used in combination with statins can be in immediate release or modified release formulations. For the present invention, combination will include sequential or simultaneous or co-administered or combined administration.

The pharmaceutical composition of the invention can be formed by various methods known in the art such as by dry granulation, wet granulation, melt granulation, direct compression, double compression, extrusion spheronization, layering and the like.

The modified release formulation of the present invention provides in-vivo plasma concentration of the statins with decreased systemic concentration and increased hepatic bioavailability. The decrease in systemic concentration and increased hepatic bioavailability reduces the major side effects of statins.

It is known that rosuvastatin was first proposed to market at doses ranging from 10 to 80 mg. However, the original application revealed safety concerns for 80 mg dose. Data presented by sponsor showed that the development of severe myopathy or rhabdomyolysis requiring hospitalization for IV hydration occurred at an incidence only at the 80 mg dose. The incidences of CK elevations >10×ULN and myopathy in clinical trials of rosuvastatin 5 to 40 mg were between 0.2 to 0.4% and 0.1 to 0.2% respectively. Data from the clinical trials in this application show that patients receiving rosuvastatin had an increased rate of developing proteinuria with and without hematuria. Proteinuria was most pronounced at the 80 mg dose.

The incidence of myopathy, in clinical trials, of rosuvastatin 5 to 40 mg, was between 0.1 to 0.2%, which are similar to rates seen with other currently approved statins. The risk of muscle and renal toxicity appear dose related and are clearly evident at 80 mg dose. Rosuvastatin levels more than 50 ng/ml in plasma developed muscle and or renal toxicity. Only a few patients treated with 40 mg rosuvastatin (2%) had drug levels within this range and greater proportion of patients treated with 80 mg (33%) achieved drug levels greater than 50 ng/ml. Further in certain clinical situations which may increase drug level require careful consideration as patients in these settings may be exposed to drug levels beyond what is typical for the 20 and 40 mg doses as a consequence of drug interaction or use in patients with severe renal disease or in geriatric patients.

Thus, the modified release formulations of the present invention, provide modified release statin, most preferably rosuvastatin, in vivo when given once daily. Maximum concentrations (Cₘₐₓ) of statin, most preferably rosuvastatin, in plasma are statistically lower than the immediate release formulation upon oral administration.

After oral administration of a pharmaceutical composition according to the present invention it is contemplated that the plasma concentration versus time profile show an extended period of time in which the plasma concentration is maintained within the therapeutic window (i.e. the plasma concentration leads to a therapeutic effect) without leading to serious unwanted adverse effects. Thus, a reduction in systemic exposure is observed. In a specific embodiment, it may be of interest to provide a pharmaceutical composition comprising rosuvastatin or a derivative or analogue thereof together with one or more pharmaceutically acceptable excipient, wherein the composition upon oral administration to a mammal in need thereof release rosuvastatin or a derivative or analogue thereof in a controlled manner and exhibits a Cₘₐₓ lower than the immediate release formulation, shows reduced number of peaks in the plasma concentration vs time curve thereby reducing the total systemic exposure. It is further contemplated that the formulation of the present invention would have better efficacy compared to the immediate release formulation.

The foregoing examples are illustrative embodiments of the invention and are merely exemplary. A person skilled in the art may make variations and modifications without deviating from the spirit and scope of the invention. All such modifications and variations are intended to be included within the scope of the invention.

### EXAMPLES

### Example 1

| | % w/w |
|---|---|
| Ingredients | |
| Rosuvastatin Calcium | 2 |
| HPMC | 15 |
| Croscarmellose Sodium | 15 |
| Microcrystalline Cellulose | 20 |
| Lactose | 47 |
| Magnesium stearate | 1 |
| Coating | |
| Film Coating | q.s |

Procedure:
1) Rosuvastatin Calcium was sifted through sieve.
2) HPMC, Crosscarmellose Sodium, Microcrystalline Cellulose & Lactose were sifted together through sieve.
3) Steps 1 was mixed with step 2 for 15 min.
4) Magnesium Stearate was sifted through sieve and step 3 was lubricated using magnesium stearate
   a) Lubricated blend of step 4 was compressed
5) Compressed tablets were film coated.

### Example 2

| | % w/w |
|---|---|
| Ingredients | |
| Rosuvastatin Calcium | 2 |
| HPMC | 2 |
| Croscarmellose Sodium | 10 |
| Mannitol | 15 |
| Microcrystalline Cellulose | 29 |
| Lactose | 40.5 |
| Magnesium Stearate | 1.5 |
| Coating | |
| Eudragit | 80 |
| Triethyl citrate | 20 |
| Isopropyl alcohol | q.s |
| Dichloromethane | q.s |

Procedure:
1) Sift the Lactose (75% of Stated Amt) Along with Rosuvastatin Calcium.
2) Sift the Magnesium Stearate
3) Lubricate the Blend of Step 1 using step 2 for 5 min.
4) Prepare the slugg of blend of step.
5) Deslugged the slugg of step 4. by crushing through required sieves.
6) Sift the HPMC, Croscarmellose Sodium, and Mannitol, MCC, & remaining qty of Lactose mix well with step 5 and sift together.
7) Sift the Magnesium Stearate through
8) Lubricate the blend of step 7.
9) Compress the blend of step 8.

Coating:

Take Sufficient qty of IPA & DCM (1:1) add req. qty of Eudragit, and TEC dissolved in stated solvent with continuous stirring. Coat the core tablets.

### Example 3

| | % w/w |
|---|---|
| Ingredients | |
| Rosuvastatin Calcium | 2 |
| Xanthan Gum | 17 |
| Microcrystalline Cellulose | 32.5 |
| Aerosil | 5 |
| Lactose | 47 |
| Magnesium Stearate | 1 |
| Coating | |
| Film Coating | q.s |

Procedure:

1) Sift Rosuvastatin Calcium along with 85% of Lactose

2) Sift magnesium stearate and lubricate the above blend of step 1 with it.

3) Compress the above blend.

4) Crushed the above slug and pass it through suitable seive

5) Sift the remaining qty of Lactose, MCC, Xanthan gum, Aerosil.

6) Mix the Blend of step 4 along with blend of step 5.

7) Sift the magnesium stearate and lubricate the blend of step 6 with it.

8) Compress the blend of step 7 suitable punch Tablets were film coated.

### Example 4

| | % w/w |
|---|---|
| Ingredients | |
| Rosuvastatin Calcium | 2 |
| HPMC | 11 |
| Croscarmellose Sod. | 15 |
| Microcrystalline Cellulose | 27 |
| Aerosil | 0.5 |
| Lactose | 43 |
| Magnesium Stearate | 1.5 |
| Coating | |
| Film Coating | q.s |

Procedure:

1) Sift Rosuvastatin Calcium along with 85% of Lactose

2) Sift magnesium stearate and lubricate the above blend of step 1 with it.

3) Compress the above blend.

4) Crushed the above slug and pass it through suitable seive

5) Sift the remaining qty of Lactose, Microcrystalline Cellulose, HPMC, Aerosil.

6) Mix the Blend of step 4. along with blend of step 5.

7) Sift the magnesium stearate and lubricate the blend of step 6 with it.

8) Compress the blend of step 7 suitable punch Tablets were film coated.

In-Vitro Dissolution Study

Following in-vitro dissolution method was used.

A solution of 900 ml of a 0.001N HCL (pH3.5) was used as dissolution medium. The apparatus contained basket (USP I) and rotated at a speed of 100 rpm. The tablet formulation was placed in the apparatus and dissolution was periodically measured. The in vitro dissolution studies of Example 4 is as follows:

| Time (hr) | Cumulative % Drug Release |
|---|---|
| 2 | About 10 to about 50 |
| 4 | About 20 to about 70 |
| 6 | About 30 to about 80 |
| 8 | About 40 to about 90 |
| 24 | NLT 75 |

In-Vivo Bioequivalence Study

An Open label, balanced, randomized, two-treatment, two-period, two-sequence, single dose, crossover relative bioavailability study of Rosuvastatin Calcium SR Tablets 20 mg (once daily) prepared according to example 4 comparing with Crestor® 20 mg (containing Rosuvastatin 20 mg IR) Tablets (once daily) manufactured for AstraZeneca Pharmaceuticals LP in ten normal healthy, adult, male, human subjects under fed condition was carried out.

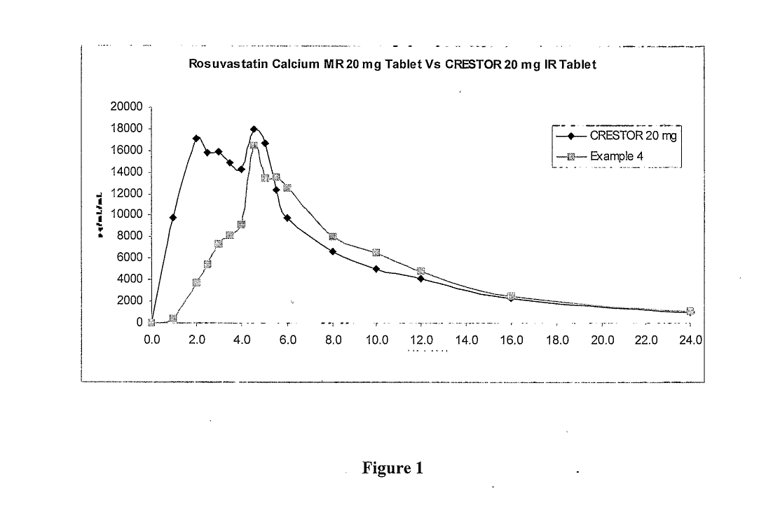

The in-vivo bioequivalence study shows the result as shown in table below. And FIG. 1.

| | AUC (0-t) | AUC (0-∞) | Cₘₐₓ | Tₘₐₓ | T½ |
|---|---|---|---|---|---|
| | ng · hr/ml | ng · hr/ml | ng/ml | Hour | Hour |
| Example 4 | 13.1 | 13.5 | 18.9 | 4.5 | 7.10 |
| Crestor ® | 15.4 | 16.0 | 23.7 | 2.5 | 11.43 |
| P-Value | 0.1142 | 0.0888 | 0.1491 | — | — |
| (ANOVA) | | | | | |

These results show that the Cₘₐₓ of rosuvastatin formulation is statistically lower when compared to the marketed formulation, Crestor®. Further, the drug plasma concentration vs time profile (FIG. 1) for modified release formulation of the present invention and Crestor® clearly depicts that in Crestor®, there are two sharp peaks indicating that the first peak is due to systemic absorption of the drug and second peak due to enterohepatic recirculation. However, the formulation of the present invention shows only one sharp peak, which may be due to eneterohepatic recirculation. This shows the total systemic exposure of rosuvastatin in systemic circulation is reduced as compared to the immediate release Crestor®. This probably reduces the adverse effects associated with the systemic absorption of rosuvastatin. It indicates that the modified release rosuvastatin formulation of the present invention shows higher hepatic bioavailability and lower systemic bioavailability, thereby reducing adverse effects. Further, there is statistically lower Cₘₐₓ for modified release formulations of rosuvastatin as compared to immediate release formulation, Crestor®.

1: A modified release pharmaceutical formulation comprising a therapeutically effective amount of HMG CoA reductase inhibitor or a pharmaceutically acceptable salt(s), polymorph(s), solvate(s), hydrate(s), prodrug or metabolite thereof, one or more release modifying agent(s) and one or more pharmaceutically acceptable excipient(s), wherein the modified release formulation provides reduced incidence of adverse effects and improved efficacy when compared to the immediate release formulation upon oral administration.

2: A modified release pharmaceutical formulation according to claim 1, wherein the modified release formulation provides reduced incidence of rhabdomyolysis, myopathy and renal toxicity as compared to immediate release composition upon oral administration.

3: A modified release pharmaceutical formulation according to claim 1, wherein the HMG CoA reductase inhibitor is selected from the group comprising lovastatin, pravastatin, atorvastatin, nivastatin, rosuvastatin, cerivastatin, pitavastatin and simvastatin.

4: A modified release pharmaceutical formulation according to claim 1, wherein the release modified polymers are hydrophilic or hydrophobic or combinations thereof.

5: A modified release pharmaceutical formulation comprising a therapeutically effective amount of rosuvastatin or a pharmaceutically acceptable salt(s), polymorph(s), solvate(s), hydrate(s), prodrug or metabolite thereof, one or more release modifying agent(s) and one or more pharmaceutically acceptable excipient(s), so that upon oral ingestion, maximum peak concentrations of the rosuvastatin are statistically lower than those produced by an immediate release pharmaceutical composition.

6: A modified release pharmaceutical formulation comprising a therapeutically effective amount of rosuvastatin or a pharmaceutically acceptable salt(s), polymorph(s), solvate(s), hydrate(s), prodrug or metabolite thereof, one or more release modifying agent(s) and one or more pharmaceutically acceptable excipient(s), so that upon oral ingestion, time to reach maximum peak concentrations of the rosuvastatin are statistically longer than those produced by an immediate release pharmaceutical composition.

7: A modified release pharmaceutical formulation comprising a therapeutically effective amount of rosuvastatin or a pharmaceutically acceptable salt(s), polymorph(s), solvate(s), hydrate(s), prodrug or metabolite thereof, one or more release modifying agent(s) and one or more pharmaceutically acceptable excipient(s), so that upon oral ingestion, area under the concentration-time curve of the rosuvastatin are statistically lower than those produced by an immediate release pharmaceutical composition.

8: A modified release pharmaceutical formulation comprising a therapeutically effective amount of rosuvastatin or a pharmaceutically acceptable salt(s), polymorph(s), solvate(s), hydrate(s), prodrug or metabolite thereof, one or more release modifying agent(s) and one or more pharmaceutically acceptable excipient(s), wherein the modified release formulation provides increased hepatic bioavailability and decreased systemic bioavailability when compared to the immediate release formulation upon oral administration.

9: A modified release pharmaceutical formulation comprising a therapeutically effective amount of rosuvastatin or a pharmaceutically acceptable salt(s), polymorph(s), solvate(s), hydrate(s), prodrug or metabolite thereof, one or more release modifying agent(s) and one or more pharmaceutically acceptable excipient(s), wherein the total systemic exposure of rosuvastatin in systemic circulation is reduced when compared to the immediate release formulation upon oral administration.

10: A modified release pharmaceutical formulation comprising a therapeutically effective amount of rosuvastatin or a pharmaceutically acceptable salt(s), polymorph(s), solvate(s), hydrate(s), prodrug or metabolite thereof, one or more release modifying agent(s) and one or more pharmaceutically acceptable excipient(s), wherein the composition upon oral administration to a mammal in need thereof releases rosuvastatin in a controlled manner thereby minimizing or avoiding the first peak concentration as compared to the two peak concentrations observed with immediate release formulation.

11: A modified release pharmaceutical formulation according to claim 10, wherein the first peak concentration is avoided as compared to two peak concentrations in the immediate release formulation.

12: A modified release pharmaceutical formulation comprising a therapeutically effective amount of rosuvastatin or a pharmaceutically acceptable salt(s), polymorph(s), solvate(s), hydrate(s), prodrug or metabolite thereof, one or more modified release polymer(s) and one or more pharmaceutically acceptable excipient(s); wherein about 10-50% of drug is released in about 2 hours, about 20-70% of drug is released in about 4 hours, about 40-90% of drug is released in about 8 hrs, and more than about 75% of drug is released in about 24 hours.
